(19) United States
(12) Patent Application Publication
(10) Pub. No.: US 2022/0095607 A1
(43) Pub. Date: Mar. 31, 2022

(54) PRESERVATION OF VASCULARIZED COMPOSITE ALLOGRAFTS

(71) Applicant: The General Hospital Corporation, Boston, MA (US)

(72) Inventors: Mustafa Korkut Uygun, Newton, MA (US); Laura Burlage, Amsterdam (NL); Shannon N. Tessier, Framingham, MA (US); Curtis L. Cetrulo, Cohasset, MA (US); Alexandre G. Lellouch, Harrison, NY (US); Reinier Johan De Vries, Boston, MA (US); Mehmet Toner, Charlestown, MA (US)

(21) Appl. No.: 17/428,593

(22) PCT Filed: Feb. 5, 2020
(86) PCT No.: PCT/US2020/016840

§ 371 Date: Aug. 4, 2021

Related U.S. Application Data
(60) Provisional application No. 62/801,284, filed on Feb. 5, 2019.

(51) Int. Cl. A01N 1/02

(52) U.S. Cl. CPC A01N 1/0221; A01N 1/0247; A01N 1/0284; A01N 1/0226

(57) ABSTRACT

This disclosure relates to subnormothermic machine perfusion formulations for ex vivo preservation of allografts, and methods of use thereof.

### CLAIM OF PRIORITY

This application claims the benefit of U.S. Provisional Application Ser. No. 62/801,284, filed on Feb. 5, 2019. The entire contents of the foregoing are incorporated herein by reference.

### FEDERALLY SPONSORED RESEARCH OR DEVELOPMENT

This invention was made with Government support under Grant Numbers DK096075, DK107875, DK114506, and AI124835 awarded by the National Institutes of Health, and Grant Number W81XWH-17-1-0680 awarded by United States Department of Defense. The Government has certain rights in the invention.

### TECHNICAL FIELD

This disclosure relates to subnormothermic perfusion formulations for ex vivo preservation of allografts, and methods of use thereof.

### BACKGROUND

Vascularized composite allotransplantation (VCA) remains the most advanced treatment option to restore motor function and aesthetics in patients living with devastating disfigurements. To date, worldwide more than 200 patients have benefited from VCA, the majority receiving hand/upper extremity or face transplants (Burlage L. C. et al. Advances in machine perfusion, organ preservation, and cryobiology: potential impact on vascularized composite allotransplantation. Curr Opin Organ Transplant 2018; 23:561-567). In all fields of transplantation, graft viability prior to transplantation is inextricably linked to post-transplant success. Minimization of graft injury prior to transplantation is therefore key to improve outcomes in VCA (Kueckelhaus M. et al. Vascularized composite allotransplantation: current standards and novel approaches to prevent acute rejection and chronic allograft deterioration. Transpl Int 2016; 29:655-662). The current standard method of graft preservation is based on cooling the graft in a cold preservation solution (4 degrees Celsius) on ice in a specialized media (typically University of Wisconsin (UW) solution or Histidine-tryptophan-ketoglutarate (HTK) solution), referred to as static cold storage (SCS). The significant drop in temperature lowers the metabolic rate of the tissue, which enable the graft to temporarily cope with the absence of oxygen and nutrients. Muscle cells (the dominant tissue type as per quantity in most VCA grafts) are, however, highly metabolic active which allows only for an extremely limited ischemia time; irreversible cell damage already occurs after as little as 4 hours of ischemia (Blaisdell F W. The pathophysiology of skeletal muscle ischemia and the reperfusion syndrome: a review. Cardiovasc Surg 2002; 10:620-630). Permanent ischemic injury occurs within 2-4 hours of warm ischemia and 6-8 hours of cold ischemia in skeletal muscle, which is abundant in amputated limbs.

Moreover, upon reperfusion, the sudden abundance of oxygen will aggravate cell damage even more, initiating reactive oxygen species (ROS) formation and intracellular calcium influx leading to mitochondrial dysfunction and eventually cell death. Apoptotic and necrotic muscle cells ultimately trigger the immune system, affecting both early and long-term graft function (Landin L. et al. Ann Plast Surg 2011; 66:202-209; Murata S. et al. Transplantation 2004; 78:1166-1171; Panizo A. et al. Transplant Proc 1999; 31:2550-2551).

### SUMMARY

The present disclosure relates to methods of subzero preservation of biological tissue samples, such as vascularized composite allografts from mammals, e.g., humans. The present disclosure is based, at least in part, on the development of methods and compositions for ex vivo sub-zero non-freezing (SZNF) preservation, which chills the tissue to temperatures below freezing point (e.g., about −5° C.) without any phase change, slows down the metabolic and degradation processes beyond what is currently possible at ice-cold temperatures (e.g., about +4° C., e.g., SCS), and extends the overall duration of preservation. The methods can include the use of growth factors, oncotic agents and/or a multi-step protocol as described herein to minimize swelling and enhance VCA viability. As shown herein, using these methods viable biological tissue samples can be preserved for extended periods of time.

In one aspect, the present disclosure relates to methods for preserving a biological tissue sample, the method including: (a) perfusing the biological tissue sample with a sub-normothermic perfusion solution including one or more cryoprotective agents, one or more oxygen carrier agents, one or more growth factors, and one or more vasodilators, at a sub-normothermic temperature; (b) perfusing the biological tissue sample with a subzero non-freezing preservation solution including at least one or more cryoprotective agents, at a hypothermic temperature; (c) optionally placing the perfused biological tissue sample in a container and sealing the container; and (d) cooling the biological tissue sample in the container to a subzero temperature without freezing the sample, thereby preserving the biological tissue sample at the subzero temperature.

In some embodiments, the method also includes warming the biological tissue sample to a hypothermic temperature; perfusing the biological tissue sample with a recovery solution including one or more cryoprotective agents and one or more oxygen carrier agents at a sub-normothermic temperature; and warming the biological tissue sample to a normothermic temperature, thereby recovering the preserved biological tissue sample for use.

In another embodiment, the method includes, preferably prior to step (a), removing hair from the biological tissue sample, sufficient to avoid ice crystal formation within the biological tissue sample or the perfusion solution. Further, the method can also include removing the hair from the biological tissue sample by contacting the biological tissue sample with a chemical depilatory agent. In yet other embodiments, the sub-normothermic perfusion solution includes one or more cryoprotective agents selected from polyethylene glycol (PEG) and 3-OMG, in a skeletal muscle cell growth medium.

Still further, in other embodiments, the hypothermic temperature is between 0° C. and 12° C. In certain embodiments, the hypothermic temperature is about 4° C. In yet other embodiments, the sub-normothermic temperature is between 12° C. and 35° C. In another embodiment, the sub-normothermic temperature is about 21° C. In some embodiments, the normothermic temperature is between about 35° C. and 40° C. In various embodiments, the normothermic temperature is about 37° C. In some embodiments, the subzero temperature is about −4° C. In another embodiment, the subzero temperature is below about −4° C., e.g., below −5° C., −6° C., −7° C., −8° C., −9° C., −10° C., −11° C., −12° C., −13° C., −14° C., −15° C., −16° C., −17° C., −18° C., −19° C., −20° C., −25° C., −30° C., −35° C., or −40° C.

In certain embodiments, the removal of sufficient air from the container results in elimination or reduction of one or more liquid-air interfaces in the container, thereby reducing or eliminating formation of ice crystals. In various embodiments, the biological tissue sample remains unfrozen when cooled to a subzero temperature. In some embodiments, the biological tissue sample is a vascular composite allograft. In various embodiments, the vascular composite allograft is a donor vascular composite allograft for vascular composite allograft transplantation. In some embodiments, the biological tissue sample is obtained from a human, a primate, or a pig. In another embodiment, the vascular composite allograft is at least a portion of a portion of a limb (e.g., all or part of an upper extremity including all or part of one or more digits, hand, nails, forearm, elbow, and/or upper arm, or all or part of a lower extremity including legs, ankles, feet, and one or more toes), face (e.g., all or part of a face including eye, periorbital tissue/eyelids, ear, nose, and/or a lip or lips), larynx, trachea, abdominal wall, genitourinary tissue (e.g., labia, a penis and/or urethra), uterine tissue (e.g., endometrium), solid organ, or a combination thereof.

In some embodiments, the recovery solution includes one or more of polyethylene glycol (PEG), an oxygen carrier agent, a prostaglandin, an albumin, skeletal muscle cell growth medium. In various embodiments, the sub-normothermic perfusion solution and the recovery preservation solution include: between 50 mL and 200 mL oxygen carrier agent per 500 mL; between 1 g and 20 g albumin per 500 mL; between 1 g and 50 g 35 kDa PEG per 500 mL; between 0.02 μL/min and 2 μL/min prostaglandin (10 μg/mL); and skeletal muscle cell growth medium. For example, in some embodiments, the sub-normothermic perfusion solution and the recovery solution can include: about 125 mL oxygen carrier agent per 500 mL; about 10 g albumin per 500 mL; about 15 g 35 kDa PEG per 500 mL; about 0.2 μL/min prostaglandin (10 μg/mL); and skeletal muscle cell growth medium. In some embodiments, both the sub-normothermic perfusion solution and the recovery solution are hyperosmolar.

In various embodiments, the sub-normothermic perfusion solution and the recovery solution includes: between 50 U and 150 μL insulin per 500 mL; between 1 mg and 20 mg dexamethasone per 500 mL; between 0.1 mL and 5 mL heparin per 500 mL; between 1 mL and 10 mL antibiotic (5000 U/ml) per 500 mL; between 1 mL and 10 mL L-glutamine per 500 mL; and between 50 μL and 150 μL immune suppressant. For example, in some embodiments, the sub-normothermic perfusion solution and the recovery solution include: about 100 μL insulin per 500 mL; about 8 μg dexamethasone per 500 mL; about 1 mL heparin per 500 mL; about 2 mL antibiotic (5000 U/ml) per 500 mL; about 5 mL L-glutamine per 500 mL; and about 100 μL immune suppresant 500 mL. In some embodiments, the antibiotics are penicillin and/or streptomycin.

In some embodiments, steps (a) and (b), combined, are performed for a duration of approximately 2 hours. In various embodiments, steps (d) and (e), combined, are performed for a duration of approximately 24 hours. In yet another embodiment, step (f) is performed for a duration of approximately 1 hour. In some embodiments, the biological tissue sample is preserved at the subzero temperature for more than 12 hours, e.g., more than 13, 14, 15, 16, 17, 18, 19, 20, 21, 22, 23, or 24 hours.

In various embodiments, biological tissue sample is viable after being recovered from subzero preservation, as determined by measuring one or more of a tissue adenosine triphosphate (ATP) to adenosine monophosphate (AMP) ratio, a tissue ATP to adenosine diphosphate (ADP) ratio, lactate levels, potassium concentration, terminal deoxynucleotidyl transferase dUTP nick end labeling (TUNEL), swelling and weight gain, percentage of edema, vascular resistance, oxygen consumption, lactic acid dehydrogenase (LDH) levels, and ischemia.

In some embodiments, the sub-normothermic perfusion solution and the recovery solution include a growth factor. In various embodiments, the oxygen carrier agent is an acellular oxygen carrier agent. In certain other embodiments, the acellular oxygen carrier agent is a hemoglobin-based oxygen carrier (HBOC) or a perfluorocarbon-based oxygen carrier (PFC). In another embodiment, the oxygen carrier agent is a cellular oxygen carrier, preferably red blood cells.

In one aspect, the present disclosure relates to systems for subzero preserving a biological tissue sample. For example, the system can include a pump; a solution reservoir; a heat exchanger; a hollow fiber oxygenator; a jacketed bubble trap; a pressure sensor; a tubing that serially connects the pump, the solution reservoir, the heat exchanger, the hollow fiber oxygenator, the jacketed bubble trap, and the pressure sensor; and a computer control unit that operates the system to perform any of the perfusion steps described herein.

In another aspect, the present disclosure relates to sub-normothermic perfusion solutions for preconditioning a biological tissue sample for subzero preservation. For example, the solution can include, per 500 mL volume: between 50 mL and 200 mL oxygen carrier agent; between 1 g and 20 g albumin; between 1 g and 50 g 35 kDa PEG; between 0.02 μL/min and 2 μL/min prostaglandin (10 μg/mL); and skeletal muscle cell growth medium. In one embodiment, the perfusion solution includes about 125 mL oxygen carrier agent per 500 mL; about 10 g albumin per 500 mL; about 15 g 35 kDa PEG per 500 mL; about 0.2 μL/min prostaglandin (10 μg/mL); and skeletal muscle cell growth medium.

In certain embodiments, the perfusion solution includes a growth factor. In some embodiments, the growth factor is fibroblast growth factor, basic epidermal growth factor, or a combination thereof. In various embodiments, the growth factor is platelet derived growth factor, insulin-like growth factor, vascular endothelial growth factor, hepatocyte growth factor, tumor necrosis growth factor, an interleukin, an interferon, a colony-stimulating factor, or any combination thereof.

In yet another embodiment, the perfusion solution includes a growth factor at a concentration ranging from about 10 ng/mL to about 1 mg/mL. In some embodiments, the oxygen carrier agent is an acellular oxygen carrier agent. In various embodiments, the oxygen carrier agent is a hemoglobin-based oxygen carriers (HBOC) or a perfluorocarbon-based oxygen carrier (PFC). In various other embodiments, the oxygen carrier agent is a cellular oxygen carrier, preferably including red blood cells.

Disclosed herein, in certain embodiments, are optimized methods and compositions to preserve organ and/or tissue grafts intended for vascularized composite allotransplantation in a host or recipient mammal comprised of ex vivo vascular perfusion of the organ or tissue graft with a non-freezing perfusate at high subzero temperatures followed later by warm machine perfusion of the organ or tissue grafts for transplantation.

The term “subzero preservation” as used herein refers to the preservation of biological tissue samples at temperatures below the freezing temperature of water (i.e., 0° C.). Subzero preservation has the potential to extend the storage limits of biological tissue samples such as organs, as the metabolic rate halves for every 10° C. reduction in temperature, thereby reducing the rate of biological tissue sample deterioration.

The term “subzero non-freezing preservation” as used herein refers to cooling a substance such as a liquid or a liquid within a biological tissue to a temperature below its melting point (or freezing point) without solidification or crystallization (e.g., ice crystal formation). Under normal atmospheric conditions, ice transitions to water at 0° C., i.e., the melting point. Nevertheless, the observed freezing temperature for pure water is usually below the melting point.

The term “liquid-air interface” or “air-liquid interface” as used herein refers to the boundary between a liquid and a gas (or biological tissue and gas) that can exist, for example, in a container that is holding a biological tissue sample being preserved. In general, the likelihood of ice crystal formation in biological tissue samples is greater for biological tissue samples having larger dimensions.

As used herein, the term “about” means plus or minus 10%.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows an example ex vivo subnormothermic machine perfusion system.

FIGS. 2A-F show an overview of perfusion parameters measured during subnormothermic machine perfusion.

FIG. 3 shows energy charge ratio values measured in the vascularized composite allografts perfused with different perfusion solutions.

FIG. 4 shows representative muscle histology images of vascularized composite allografts after 6 hours of subnormothermic machine perfusion with different perfusion solutions.

FIG. 5 shows images of heterotopic hind limb transplant grafts on post-operative days (POD) 0, 7, 15, 21 and 30.

FIG. 6A shows transplant survival rates of rodent recipients of vascularized composite allografts perfused with different perfusion solutions.

FIG. 6B shows the various causes of death among the rodent recipients of vascularized composite allografts perfused with different perfusion solutions.

FIG. 7 provides an exemplary protocol for VCA preservation. This protocol includes a 2 hour loading phase SNMP. During this phase grafts were perfused with an exemplary Sub-Normothermic Perfusion Solution based on PromoCell skeletal muscle media, 3-OMG, 3% BSA, 5% PEG, epidermal and fibroblast growth factors, heparin, insulin, antibiotics, L-glutamine, dexamethasone, hydrocortisone, prostaglandin. Afterwards, limbs were cooled to about 4° C. with cold flush of the same solution (30 min). Then, the limbs were flushed or perfused with and submerged in the subzero non-freezing preservation solution including HTK and PEG before storing them in a chiller and lowering the temperature to about −5° C. After subzero non-freezing preservation, limbs were recovered using again SNMP with a recovery solution similar to the loading phase but without 3-OMG and with an oxygen carrier, e.g., a cellular or acellular oxygen carrier or red blood cells.

FIG. 8 is an image of a hind limb during subzero non-freezing preservation. During the loading phase, the graft was perfused with a Sub-Normothermic Perfusion Solution followed by a cold flush (4 degrees Celsius) of the same solution and subsequently a flush with the ‘SZNF solution’ (4 degrees Celsius). The graft is stored in a non-freezing preservation solution and hanged in a basin with the anti-freeze solution. The temperature of the chiller was gradually lowered at a rate of 0.1 degree Celsius per minute. Once the temperature had reached minus 5 degrees Celsius, the limb was stored for 24 hours. After 24 hours of SZNF, the temperature of the chiller was gradually rewarmed. Once the temperature in the chiller has reached 4 degrees Celsius, the limb was connected to the perfusion system and perfused for 1 hour using a recovery solution.

### DETAILED DESCRIPTION

The present disclosure relates to improved protocols and/or perfusion solutions that avert freezing and crystal formation in the cells and tissues of tissue samples, e.g., vascularized composite allografts (VCAs). The examples below show that vascularized composite allografts subjected to a multistep protocol including ex vivo sub-normothermic machine perfusion (SNMP) using an oxygen carrier, growth factors, and oncotic agents to reduce swelling results in superior tissue preservation compared to conventional static cold preservation. Moreover, the examples below show transplantation of these preserved VCAs is feasible and can show promising results (e.g., preserved tissues showed decreased edema, decreased ischemia, increased oxygen consumption rate, and an increased energy charge ratio compared to controls and/or non-oxygen carrier-preserved tissues).

Embodiments described below include subzero non-freezing preservation protocols and/or perfusion or preservation solutions featuring oxygen carriers, specialized cell media, oncotic agents, and growth factors designed to preserve VCAs and enhance their viability. In some embodiments, a distinct advantage of the subzero non-freezing preservation methods and solutions of the disclosure is that they improve the viability of preserved tissues by, for example, reducing edema or weight gain in preserved biological tissue samples as compared to biological tissue samples preserved via other methods (e.g., preservation methods used to preserve organs or standard static cold preservation techniques). Reducing edema or weight gain in preserved biological tissue samples is vital given that increased levels of edema or weight gain (e.g., greater than about 20%) can lead to transplant or graft failure or at least reduce viability of the preserved biological tissue sample.

In some embodiments, an additional advantage of the subzero non-freezing preservation methods and solutions of the disclosure is that they improve the viability of preserved tissues by, for example, increasing the total oxygen consumption in preserved biological tissue samples as compared to biological tissue samples preserved via other methods (e.g., preservation methods used to preserve organs or standard static cold preservation techniques). Increasing the total oxygen consumption in preserved biological tissue samples translates into increasing the viability and function of the tissue, thereby facilitating a successful transplantation and post-operative outcome for the tissue graft recipient.

In some embodiments, yet another advantage of the subzero non-freezing preservation methods and solutions of the disclosure is that they improve the viability of preserved tissues by, for example, increasing an energy charge ratio in preserved biological tissue samples as compared to biological tissue samples preserved via other methods (e.g., preservation methods used to preserve organs or standard static cold preservation techniques). The energy charge ratio can be determined by measuring the levels of adenosine triphosphate (ATP), adenosine diphosphate (ADP), and adenosine monophosphate (AMP), which are energetic co-factors. In some embodiments, and as disclosed in the Examples, the energetic ration can be defined by Equation 1 below:

Energy Charge Ratio=(ATP+0.5*ADP)/(ATP+ADP+AMP). Equation 1:

In some embodiments, the energy charge ratio essentially reflects the preserved energy status of the preserved biological tissue sample. In some embodiments, preserved energy status is critical for a successful post-transplant outcome. (See e.g., Bruinsma B G, Avruch J H, Sridharan G V, et al. Transplantation 2017; 101:1637-1644). Thus, an increase in the energy charge ratio of preserved biological tissue samples improves a post-operative outcome for the tissue graft recipient and can lead to a successful graft transplantation.

In some embodiments, an additional advantage of the subzero non-freezing preservation methods of the disclosure is that it allows preservation at high subzero storage temperature (approximately −4° C., for example, −5° C. to −3° C., −6° C. to −2° C., or −7° C. to −1° C.), while avoiding phase transitions and consequent lethal ice-mediated injury (Bruinsma, B. G. & Uygun, K. Curr. Opin. Organ Transplant. 22, 281-286 (2017); Berendsen, T. A. et al. Nat. Med. 20, 790-793 (2014); Bruinsma, B. G. et al. Nat. Protoc. 10, 484-494 (2015)), as well as toxicity of most common CPAs. For example, in some embodiments, subzero non-freezing can allow preservation at lower temperature than high subzero storage temperature (e.g., below −4° C., −5° C., −6° C., −7° C., −8° C., −9° C., −10° C., −11° C., −12° C., −13° C., −14° C., −15° C., −16° C., −17° C., −18° C., −19° C., −20° C., −25° C., −30° C., −35° C., −40° C., or even lower temperature). Subzero non-freezing preservation can include supercooling. In some embodiments, the methods can include using freezing point depressors and/or higher pressure.

Subzero non-freezing preservation allows for extended preservation of biological tissue samples, for example, for days to months (e.g., greater than 12 hours, 18 hours, 1, 2, 3, 4, 5, 6, 7, 8, 9, 10, 20, or 30 days, greater than 1, 2, 3, 4, 5, or 6 weeks, or greater than 1, 2, 3, 4, 5, or 6 months). In some embodiments, the preservation period is less than 2, 3, 4, 5, 6, 7, 8, 9, 10, 20, or 30 days, less than 1, 2, 3, 4, 5, or 6 weeks, or less than 1, 2, 3, 4, 5, or 6 months.

The cooling rate for subzero preservation can also vary. In some embodiments, the cooling can be at a rate of <50° C./minute, e.g., <20° C./minute, <10° C./minute, <9° C./minute, <8° C./minute, <7° C./minute, <6° C./minute, <5° C./minute, <4° C./minute, <3° C./minute, <2° C./minute, <1° C./minute, <0.9° C./minute, <0.8° C./minute, <0.7° C./minute, <0.6° C./minute, <0.5° C./minute, <0.4° C./minute, <0.3° C./minute, <0.2° C./minute, or <0.1° C./minute. In some embodiments, the cooling rate is about 1° C./minute.

In some embodiments, the subzero temperature is below 0° C., e.g., below −1° C., below −2° C., below −3° C., below −4° C., below −5° C., below −6° C., below −7° C., below −8° C., below −9° C., below −10° C., below −11° C., below −12° C., below −13° C., below −14° C., below −15° C., below −20° C., below −25° C., below −30° C., below −35° C. or below −40° C. In some embodiments, the subzero temperature is above −40° C., e.g., above −35° C., above −30° C., above −25° C., above −20° C., above −15° C., above −14° C., above −13° C., above −12° C., above −11° C., above −10° C., above −9° C., above −8° C., above −7° C., above −6° C., above −5° C., above −4° C., above −3° C., above −2° C., or above −1° C.

In some embodiments, the biological tissue samples can have a volume of greater than 1 mL, 10 mL, 20 mL, 30 mL, 40 mL, 50 mL, 60 mL, 70 mL, 80 mL, 90 mL, 100 mL, 110 mL, 120 mL, 130 mL, 140 mL, 150 mL, 175 mL, 200 mL, 250 mL 300 mL, 350 mL, 400 mL, 450 mL, 500 mL, 600 mL, 700 mL, 800 mL, 900 mL, 1 L, 1.1 L, 1.2 L, 1.3 L, 1.4 L, 1.5 L, 1.6 L, 1.7 L, 1.8 L, 1.9 L, 2.0 L, 2.5 L, 3 L, 3.5 L, 4 L, 4.5 L, or 5 L. In other embodiments, the biological tissue samples can have a volume of less than 1 mL, 10 mL, 20 mL, 30 mL, 40 mL, 50 mL, 60 mL, 70 mL, 80 mL, 90 mL, 100 mL, 110 mL, 120 mL, 130 mL, 140 mL, 150 mL, 175 mL, 200 mL, 250 mL, 300 mL, 350 mL, 400 mL, 450 mL, 500 mL, 600 mL, 700 mL, 800 mL, 900 mL, 1 L, 1.1 L, 1.2 L, 1.3 L, 1.4 L, 1.5 L, 1.6 L, 1.7 L, 1.8 L, 1.9 L, 2.0 L, 2.5 L, 3 L, 3.5 L, 4 L, 4.5 L, or 5 L.

In some embodiments, the biological tissue samples can be perfused using hypothermic machine perfusion (HMP; 0-12° C.), sub-normothermic machine perfusion (SNMP; 12-35° C.), normothermic machine perfusion (NMP; >35), or using gradual rewarming whereby the temperature of the biological tissue sample is gradually raised.

In some embodiments, the hypothermic temperature can be between about 0-12° C., 1-10° C., between 2-8° C., between 3-6° C., or about 4° C.

In some embodiments, the sub-normothermic temperature can be between about 12-35° C., 15-30° C., 18-25° C., or about 21° C.

In some embodiments, the normothermic temperature can be between about 35° C. and 40° C., e.g., about 36° C., about 37° C., about 38° C., about 39° C., or about 40° C.

The present disclosure provides new methods for preservation of biological tissue samples. The methods can involve contacting, perfusing, and/or submerging the biological tissue sample with one or more of a recovery solution, perfusion solutions (e.g., a first perfusion solution and a second perfusion solution), or any other solutions as described herein in a storage solution bag or other similar containers (e.g., a surgical isolation bag), and cooling the biological tissue sample to a subzero temperature without the formation of ice crystals in cells of the tissues.

The present disclosure can be used for preserving a VCA, e.g., a mammalian, e.g., human, VCA. The methods include perfusing, contacting, or immersing the VCA with solutions, e.g., as described herein, and chilling the VCA for subzero non-freezing preservation. Methods of perfusing a VCA are known in the art. For example, perfusion can be performed by flushing or pumping a solution over or through the arteries or veins of the VCA. In some embodiments, a perfusion device (e.g., a pump or injector) can be used. Alternatively or in addition, the VCA can also be immersed within the perfusion solutions or recovery solutions. In some embodiments, the method can include multiple perfusing, contacting, or immersing steps involving multiple solutions.

The methods as described herein can also improve the outcome (e.g., viability) of preservation of biological tissue samples, or extend the length of time for which an organ can be preserved while maintaining viability for transplantation. The tissue or organs are prepared for preservation using techniques described herein. In some embodiments, the tissue or organs are obtained using art known techniques and maintained in recovery solutions appropriate for the biological tissue samples.

The methods described herein can be used to preserve biological tissue sample at a subzero temperature without freezing or ice crystal formation for various time periods, for example, for more than 12, 13, 14, 15, 16, 17, 18, 19, 20, 21, 22, 23, or 24 hours, or more than 1, 2, 3, 4, 5, 6, or 7 days, or for more than 1, 2, 3, 4, 5, or 6 months, or even longer. In some embodiments, the period is less than 12, 13, 14, 15, 16, 17, 18, 19, 20, 21, 22, 23, or 24 hours, or less than 1, 2, 3, 4, 5, 6, or 7 days, or for less than 1, 2, 3, 4, 5, or 6 months.

The methods described herein can be used for the preservation of any VCAs, e.g., mammalian, e.g., human VCAs, such as VCAs including multiple tissue types including blood vessels and other tissues such as adipose, skin, muscle, nerves, ligaments, and/or bone, e.g., osteomyocutaneous grafts, or any tissues that can be perfused through a vessel such as limbs and other vascular composite allografts. In some embodiments, the biological tissue sample can be a VCA including skin, fat, bone, muscle, ligament, tendon, artery, vein, nerve, cartilage or any combination thereof. In some embodiments, a VCA is a portion of a limb (e.g., all or part of an upper extremity including all or part of one or more digits, hand, nails, forearm, elbow, and/or upper arm, or all or part of a lower extremity including legs, ankles, feet, and one or more toes), face (e.g., all or part of a face including eye, periorbital tissue/eyelids, ear, nose, and/or a lip or lips), larynx, trachea, abdominal wall, genitourinary tissue (e.g., labia, a penis and/or urethra), uterine tissue (e.g., endometrium), or any tissues that can be perfused through a vessel such as limbs and other vascular composite allografts or a combination thereof. In some embodiments, the biological tissue sample is a solid organ or a functional portion thereof, e.g., all or part of a heart, kidney, lung, skin, ovary, pancreas, or liver, lung, skin, or bone for use in organ transplantation, where storage and transport of the organ is necessary between harvesting from an organ donor and transplantation of the organ in an organ recipient.

In some embodiments, the VCAs described herein refer to VCAs for transplantation, e.g., VCAs obtained from a VCA donor or organ donor and intended to be transplanted in a VCA recipient.

The time between the VCA harvesting and transplantation can vary, and can be more than for more than 12, 13, 14, 15, 16, 17, 18, 19, 20, 21, 22, 23, or 24 hours, or more than 1, 2, 3, 4, 5, 6, or 7 days, or for more than 1, 2, 3, 4, 5, or 6 months, or even longer. In some embodiments, the time between the organ harvesting and transplantation can be less than 12, 13, 14, 15, 16, 17, 18, 19, 20, 21, 22, 23, or 24 hours, or less than 1, 2, 3, 4, 5, 6, or 7 days, or for less than 1, 2, 3, 4, 5, or 6 months. The VCA can be a whole VCA or a portion thereof. In some embodiments, the tissue sample or organ can be a tissue for use in tissue engineering and/or regenerative medicine. In some embodiments, the tissue sample or organ can be a tissue (meat) for use in the food industry, e.g., beef, chicken, fish, poultry, goat or other meat intended for human consumption, and the methods can be used to preserve the meat until it is ready for preparation.

In some embodiments, cryoprotective agents used to pre-condition the biological tissue samples prior to subzero preservation eliminate or reduce freezing (formation of ice crystals). For example, pre-conditioning of a biological tissue sample at a hypothermic temperature (e.g., 4° C.) using any of the perfusion solutions described herein prior to subzero preservation of the biological tissue sample can eliminate or reduce freezing (formation of ice crystals), for example by reducing the melting point of the liquids within biological tissue sample. The hypothermic machine perfusion (HMP) step described herein is an example of such pre-conditioning step.

The present methods can include the stages shown in FIG. 7. Those stages can include:

(1) Obtaining a biological tissue sample from a source (e.g., a subject, a VCA donor, or an organ donor, e.g., a human or non-human subject) at a normothermic temperature (e.g., 35-40° C., e.g., about 37° C.);

(2) Cooling the biological tissue sample from a normothermic temperature (e.g., 35-40° C., e.g., about 37° C.) to a sub-normothermic temperature (e.g., about 12-35° C., or about 15-25° C., e.g., about 21° C.) in a sub-normothermic perfusion solution as described herein;

(3) Maintaining the biological tissue sample at a sub-normothermic temperature (e.g., about 12-35° C., or about 15-25° C., e.g., about 21° C.) in a sub-normothermic perfusion solution, e.g., using machine perfusion;

(4) Cooling the biological tissue sample from a sub-normothermic temperature (e.g., about 12-35° C., or about 15-25° C., e.g., about 21° C.) to a hypothermic temperature (e.g., about 2-5° C., e.g., about 4° C.), e.g., in a sub-normothermic perfusion solution;

(5) Perfusing (e.g., using hand flushing or machine perfusion) the biological sample with a subzero non-freezing preservation solution loading solution at a hypothermic temperature (e.g., 4° C.) to allow uniform perfusion of the biological tissue sample prior to subzero non-freezing preservation;

(6) Chilling (slowly enough to prevent freezing/formation of ice crystals, e.g., at a rate of about −0.1° C./minute) the biological tissue sample in a subzero non-freezing preservation solution to a subzero temperature (e.g., −2 to −7° C., e.g., −5° C.) without freezing, e.g., by a method wherein the biological tissue sample is placed in a container (e.g., an organ isolation bag), air is removed from the container to reduce liquid-air interfaces (this step results in subzero preservation of the biological tissue sample), and/or the biological tissue sample is placed in a warming and/or cooling unit having temperature regulation and rate-controlled cooling (e.g., a chiller);

(7) Maintaining the biological tissue sample at a subzero temperature in a subzero non-freezing preservation solution for a desired amount of time;

(8) Warming (slowly enough to prevent freezing/formation of ice crystals, e.g., at a rate of about −0.1° C./minute) the biological tissue sample in a subzero non-freezing preservation solution to a hypothermic temperature above freezing (e.g., about 2-5° C., e.g., about 4° C.), e.g., by a method wherein the biological tissue sample is placed in a warming and/or cooling unit having temperature regulation and rate-controlled warming (e.g., by shutting down the chiller and allowing it to warm to a hypothermic temperature above freezing, e.g., 4° C.);

(9) Perfusing the preserved biological tissue sample with a recovery solution; and

(10) Warming (rapidly or gradually) the biological tissue sample from a hypothermic temperature (e.g., about 2-5° C., e.g., about 4° C.) to a sub-normothermic temperature (e.g., about 12-35° C., or about 15-25° C., e.g., about 21° C.) in the recovery solution.

The method can optionally further comprise perfusing the biological tissue sample with a recovery solution, sub-normothermic perfusion solution, or other solution and warming the tissue sample to a normothermic temperature prior to transplantation.

Recovery of Biological Tissue Samples after Sample Acquisition

During donor procurement, heparinization of the graft is important to prevent blood clots within the graft; in some embodiments, systemic heparinization is used. After procurement, and transport (if required) at 4° C., e.g., for 1-12 hours, the biological tissue sample is “recovered” by machine perfusion at a sub-normothermic temperature (e.g., at 15-25° C., e.g., 21° C.). In some embodiments, the biological tissue sample is maintained at a normothermic temperature (e.g., 33-39° C., e.g., 37° C.) during procurement and/or transport (see Stage 1 in FIG. 7). In some embodiments, a biological tissue sample can be obtained from a subject (e.g., a mammal, e.g., a human or non-human veterinary subject, e.g., a dog, cat, horse, primate, rodent, or pig). In preferred embodiments, after procurement, sufficient hair (e.g., a portion of the hair or preferably all of the hair present on the surface of the biological tissue sample) is removed from the biological tissue sample to avoid ice crystal formation within the biological tissue sample or the perfusion solution. The hair can be removed, e.g., by shaving, waxing, or by using a chemical depilatory agent, e.g., an agent comprising one or more thioglycolic acids, thiolactic acids, and/or sulfides sufficient to dissolve keratin and remove the hair.

### Loading Phase

The loading phase can include subjecting the biological tissue sample to sub-normothermic machine perfusion with a sub-normothermic perfusion solution (see Stages 2-3 in FIG. 7), e.g., by flushing, perfusing, and/or submerging the sample with the sub-normothermic perfusion solution and cooling to a sub-normothermic temperature (e.g., 15-25° C., e.g., 21° C.). In some embodiments, this sub-normothermic machine perfusion phase (3) lasts approximately about 1 hour, e.g., 30-90 or 45-90 minutes.

In the second portion of the loading phase, the biological tissue sample is cooled (e.g., rapidly or gradually) to a hypothermic temperature (e.g., 2-5° C., e.g., about 4° C.), e.g., with flushing, perfusing, and/or submerging the sample with the same sub-normothermic perfusion solution at about 4° C. (see Stage 4 in FIG. 7). As used herein, a hypothermic temperature is above the freezing point of water, i.e., is above 0° C. Once the hypothermic temperature is reached, the sample is maintained at 4° C. for a selected time period, e.g., about 1 hour, e.g., 30-90 or 45-90 minutes (see Stage 5 in FIG. 7). In some embodiments, the biological tissue sample is flushed, perfused, and/or submerged with a subzero non-freezing preservation solution as described herein (see Stage 5 in FIG. 7) and maintained at a hypothermic temperature (e.g., 2-5° C., e.g., 4° C.).

### Subzero Non-freezing Preservation Phase

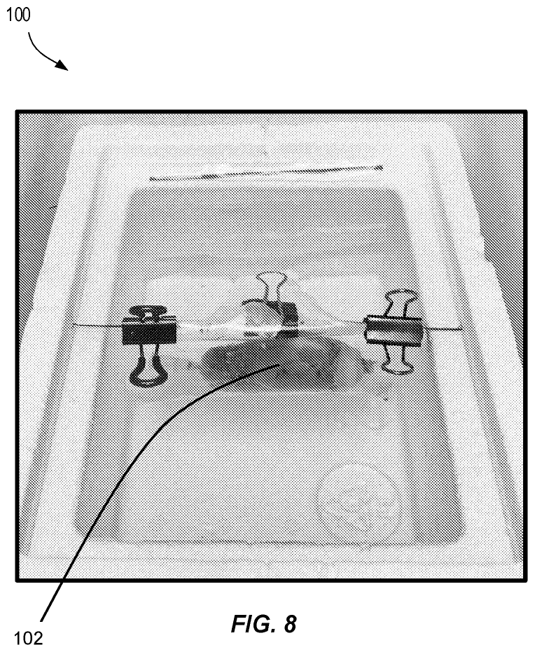

Next, the biological tissue sample, e.g., a VCA, is gradually chilled to a sub-zero temperature, without a phase change (i.e., not frozen) (see Stage 6 in FIG. 7). The biological tissue sample can be submerged within the subzero non-freezing preservation solution and chilled in a chiller at a rate sufficient to cool the biological tissue sample without formation of ice crystals, e.g., by about −0.1° C. per minute, until the biological tissue sample reaches a subzero temperature. In some embodiments, the biological tissue sample is cooled at about −0.09° C. per minute or less. In some embodiments, the biological tissue sample is cooled at up to about −0.2° C. per minute or more. During this stage (i.e., Stage 6 in FIG. 7), a container, e.g. a sealed container (e.g., a bag), containing the biological tissue sample can be placed in a fluid that can dampen vibrations for the tissue, e.g., subjected to an anti-vibration bath 100, as shown in FIG. 8. In some embodiments, the biological tissue sample within the sealed container is placed in a warming and/or cooling unit (e.g., a chiller) having a controlled temperature system and rate-controlled cooling. During the anti-vibration bath, the sealed container containing the biological tissue sample 102 is stored in the fluid that can dampen vibrations (e.g., the subzero non-freezing preservation solution) and is carefully hung in a reservoir within the cooling unit (e.g., a chiller) containing an anti-freeze solution and/or a fluid that can dampen vibrations. Since vibrations of the cooling unit (e.g., a chiller) can also initiate ice crystallization, the grafts can be hung in the anti-freeze solution to buffer the vibrations, as shown in FIG. 8.

In some embodiments, the subzero temperature is about −2 to −7° C., e.g., about −5° C. or about −4° C. In some embodiments, the subzero temperature is below about −4° C., e.g., below −5° C., −6° C., −7° C., −8° C., −9° C., −10° C., −11° C., −12° C., −13° C., −14° C., or about −15° C. In some embodiments, the subzero temperature is below about −16° C., −17° C., −18° C., −19° C., −20° C. In some embodiments, the subzero temperature is below about −25° C., −30° C., −35° C., or −40° C. For the lower temperatures, a higher osmolality subzero non-freezing preservation solution is desired to depress the freezing point, e.g., 1.5-2M of subzero non-freezing preservation solution around 20° C.

Once the temperature reaches a subzero temperature (e.g., −5° C.), the biological tissue sample is stored for about 23 hours in the subzero non-freezing preservation solution (see Stage 7 in FIG. 7). After about 23 hours of subzero non-freezing preservation of the biological tissue sample, the temperature of the reservoir, and thereby the temperature of the biological tissue sample, is gradually warmed from a sub-zero temperature to a hypothermic temperature (see Stage 8 in FIG. 7). In some embodiments, the total subzero non-freezing preservation phase (i.e., Stages 7 and 8 in FIG. 7) can last about 2 hours up to about 7 days, e.g., at least 2, 4, 6, 8, 12, 14, 16, 18 or 24 hours, and up to 6, 12, 18, 24, 30, 36, 48 hours, 3 days, 4 days, 5 days, 6, days, or 7 days, preferably 24 to 36 or 24 to 48 hours or 3-7 days. The rate at which the biological tissue sample is warmed from a sub-zero temperature to a hypothermic temperature can be about −0.1° C. per minute. In some embodiments, the biological tissue sample is warmed from a sub-zero temperature to a hypothermic temperature while in contact with the subzero non-freezing preservation solution.

### Recovery Phase

As shown in FIG. 7, the recovery phase (i.e., Stages 9 and 10 in FIG. 7) begins once the biological tissue sample reaches a hypothermic temperature (e.g., 2-5° C., e.g., 4° C.). After the biological tissue sample reaches a hypothermic temperature above freezing (e.g., 2-5° C., e.g., about 4° C.), the biological tissue sample is then warmed to a sub-normothermic temperature (e.g., about 21° C.) (see Stage 9 in FIG. 7). In some embodiments, during the recovery phase, the biological tissue sample is gradually (e.g., about 0.1° C. per minute) or rapidly warmed from a hypothermic temperature to a sub-normothermic temperature.

At this point, once the biological tissue sample has reached a sub-normothermic temperature, the biological tissue sample is connected to the perfusion system and perfused using a recovery solution (e.g., a recovery recovery solution, e.g., including a vasoactive vasodilator (e.g., prostaglandin)) at a sub-normothermic temperature (e.g., 21° C.) (see Stage 10 in FIG. 7). In some embodiments, the biological tissue sample is connected to the perfusion system and perfused using the recovery solution at a sub-normothermic temperature. In some embodiments, the biological tissue sample is connected to the perfusion system and perfused using a sub-normothermic perfusion solution at a sub-normothermic temperature. In some embodiments, the total recovery phase can last approximately 1 hour.

In some embodiments, after the recovery phase, the biological tissue sample is further gradually or rapidly warmed from a hypothermic temperature to a normothermic temperature. In some embodiments, the biological tissue sample is connected to the perfusion system and perfused using a solution, e.g., a sub-normothermic perfusion solution or recovery solution, or another solution (e.g., blood or a blood substitute), at a normothermic temperature (e.g., 37° C.).

Sub-Normothermic Perfusion Solution (SNPS)

A sub-normothermic perfusion solution for use in the present methods preferably includes one or more cryoprotective agents, one or more oxygen carrier agents, one or more oncotic agents, one or more growth factors, and one or more vasodilators, in a solution including a skeletal-muscle supporting media, e.g., Skeletal Muscle Cell Growth Medium, MUSCLE MEDIA (PromoCell), SkGM™ Skeletal Muscle Cell Growth Medium (Lonza Biologics), Primary Skeletal Muscle Growth Medium (ATCC), Skeletal Muscle Cell Growth Medium (ZenBio), or STEMLIFE SK (LifeLine Cell Tech), Skeletal Muscle Cell Growth Medium (Cell Applications/Millipore Sigma). One example is low-serum (e.g., about 6%, 5%, 4%, 3%, 2%, 1%, or less) or serum-free but chemically defined medium optional Fetuin (e.g., bovine), e.g., about 50 μg/ml; Epidermal Growth Factor (EGF, e.g., recombinant human), e.g., about 10 ng/ml; basic Fibroblast Growth Factor (e.g., recombinant human bFGF), e.g., about 1 ng/ml; insulin (e.g., recombinant human insulin), e.g., about 10 μg/ml; an immune suppressant, e.g., dexamethasone and/or hydrocortisone, e.g., about 0.4 μg/mL; and/or transferrin, e.g., about 30 μg/mL, in combination with a salt-balanced solution, including for example Ham's F12, Ham's F10, or DMEM. Exemplary formulations of SNPS are shown in Tables 1 and 2.

The perfusion solution can further contain insulin, heparin, antibiotics (e.g., penicillin-streptomycin), albumin, immune suppressants (e.g., hydrocortisone, dexamethasone), L-glutamine, and skeletal muscle cell growth medium. The perfusion solution can contain vasodilators, e.g., prostaglandins. In some embodiments, the sub-normothermic perfusion solution includes insulin, e.g., 500-100 U/L insulin, e.g., about 750 U/L insulin.

Oxygen Carrier Agents

In some embodiments, the oxygen carrier agent is an acellular or synthetic oxygen carrier agent. In some embodiments, the oxygen carrier agent is a hemoglobin-based oxygen carriers (HBOC) or a perfluorocarbon-based oxygen carrier (PFC). In some embodiments, the HBOC has a molecular weight ranging from about 100,000 to about 250,000 grams per mol (g/mol). In some embodiments, the HOBC has a molecular weight of about 201,000 g/mol. In some embodiments, the HOBC has a molecular weight of about 250,000 g/mol. In some embodiments, the sub-normothermic perfusion solution includes an acellular oxygen carrier agent with a concentration ranging from about 50 to 250 grams per liter (g/L). In some embodiments, the sub-normothermic perfusion solution includes an acellular oxygen carrier agent with a concentration of about 130 g/L. Examples include perfluorooctyl bromide (C8F17Br, perflubron); perfluorodecyl bromide (C10F21Br); perfluorodichlorooctane (C8F16Cl2); perfluorodecalin; perfluorocarbon emulsions Fluosol-DA, Oxygent and Oxyfluor. See, e.g., Spahn, Crit Care. 1999; 3(5): R93-R97. In some embodiments, the oxygen carrier agent is a Hb-based oxygen carriers (HBOCs), e.g., acellular or cellular HBOC. Acellular HBOCs include cross-linked HBOC (e.g., HEMASSIST), polymerized HBOC (e.g., HEMOPURE, POLYHEME, OXYGLOBIN, PolyHb-SOD-CAT-CA, or PolyHb-Fibrinogen) and conjugated HBOC (e.g., Hemospan or MP4). In some embodiments, the oxygen carrier agent is cellular, e.g., red blood cells (RBCs), neo red cells, hemoglobin vesicles, Liposome encapsulated actin-hemoglobin (LEAcHb); Hemoglobin-loaded polymeric nanocapsule (PNP); Cationizad HbPNP; Fe(11) porphyrin loaded dendrimer; Nanocapsule bearing a membrane made of ultrathin PEG-PLA, containing polymerized Hb and all RBC enzymes; Nanoscale hydrogel particles (NHP); Lipogel; Polymersome-encapsulated hemoglobin (PEH); Single protein nanocapsule (SNP); and Hemoglobin conjugated biodegradable polymer micelles). Hb-based RBC substitutes, e.g., human- or bovine-derived or recombinant hemoglobin (Hb) can also be used. See, e.g., Moradi et al., Clin Med Insights Blood Disord. 2016; 9: 33-41). In some embodiments, the sub-normothermic perfusion solution includes a cellular oxygen carrier agent with a concentration ranging from about 12 to 18 grams per deciliter (g/dL).

Cryoprotective Agents

The sub-normothermic perfusion solution can contain cryoprotective agents. The term “cryoprotective agents” as used herein refers to compounds or solutions of compounds, that can be used to perfuse, immerse, or contact a biological tissue sample (e.g., an organ or tissue) to preserve viability of the biological tissue sample, e.g., during storage at subzero temperatures. Exemplary cryoprotective agents include polyethylene glycol (PEG, e.g., 5-40 kD, e.g., 35 kD PEG or lower molecular weight PEGS, e.g., 8 kD, e.g., PEG 8000, PEG35000, e.g., about 0.1 to 5% w/v) and 3-O-methyl-D-glucose (3-OMG) (e.g., 0.05-0.5 M, e.g., about 0.2 M 3-OMG), and sugars such as rappinose, trehalose, and mannitol (e.g., 5-200 mM).

Oncotic Agents

The perfusion solution is hyperosmolar, i.e., contains oncotic agents (any biocompatible large molecule that will not go into the cells of the tissue, e.g., albumin, polymers or colloids such as polyethylene glycol (PEG), starches (e.g., pentastarch), dextran, or polysaccharides; the oxygen carrier and the cryoprotective agents can also act as oncotic agents) that increase the osmolality of the solution sufficiently to pull water back from the surrounding tissues to reduce swelling; in some embodiments, the final osmolality of the solution is, e.g., 250-600 mOsm/L, preferably 300-500 or 320-500 mOsm/L, e.g., 350-600 mM, e.g., 350-450 mM. See, e.g., Hoffmann R. M., Southard J. H., Belzer F. O. (1982) The use of oncotic support agents in perfusion preservation. In: Pegg D. E., Jacobsen I. A., Halasz N. A. (eds) Organ Preservation. Springer, Dordrecht.

Growth Factors

The sub-normothermic perfusion solution can also include one or more growth factors. For example, the growth factor can be basic fibroblast growth factor (FGF), epidermal growth factor (EGF), or a combination thereof. In some embodiments, the growth factor can be platelet derived growth factor (PDGF), insulin-like growth factor (IGF) (e.g., IGF-1, IGF-2), vascular endothelial growth factor (VEGF), Epidermal Growth Factor (EGF), transforming growth factor beta (TGFβ), transforming growth factor alpha (TGFα), Fibroblast Growth Factor (FGF) (e.g., basic FGF, FGF-1 FGF-2, FGF-3, FGF-7, FGF-10, FGF-22, FGF-4, FGF-5, FGF-6, FGF-8, FGF-17, FGF-18, FGF-9, FGF-16, FGF-20, FGF-19, FGF-21, FGF-23), hepatocyte growth factor, tumor necrosis factor superfamily (TNFSF) (e.g., TNF-alpha, lyphotoxin-alpha, lymphotoxin-beta, TNSF4, TNSF5, TNSF6, TNSF7, TNSF8, TNSF9, TNSF10, TNSF11, TNSF12, TNSF13, TNSF13B, TNSF14, TNSF15, TNSF18, ectodysplasin A), an anti-inflammatory interleukin (e.g., IL-2, 6), an interferon, a colony-stimulating factor (e.g., GM-CSF), or any combination thereof.

In some embodiments, the sub-normothermic perfusion solution includes a growth factor at a concentration ranging from about 10 nanograms per milliliter (ng/mL) to about 1 milligram per milliliter (mg/mL). For example, in some embodiments, the sub-normothermic perfusion solution includes a growth factor at a concentration less than 1 mg/mL, 0.9 mg/mL, 0.8 mg/mL, 0.7 mg/mL, 0.6 mg/mL, 0.5 mg/mL, 0.4 mg/mL, 0.3 mg/mL, 0.2 mg/mL, 0.1 mg/mL, 90 micrograms per milliliter (μg/mL), 80 μg/mL, 70 μg/mL, 60 μg/mL, 50 μg/mL, 40 μg/mL, 30 μg/mL, 20 μg/mL, 10 μg/mL, 1 μg/mL, 900 ng/mL, 800 ng/mL, 700 ng/mL, 600 ng/mL, 500 ng/mL, 400 ng/mL, 300 ng/mL, 200 ng/mL, 100 ng/mL, 50 ng/mL, 40 ng/mL, 30 ng/mL, 20 ng/mL, or 10 ng/mL.

Subzero Non-Freezing Preservation Solution

A sub-zero non-freezing preservative solution (i.e., the subzero non-freezing preservation solution) for use in the present methods preferably includes one or more cryoprotective agents (e.g., PEG, e.g., 1-10%, e.g., 5% PEG 35000) in an organ preservation electrolyte solution, e.g., Histidine-tryptophan-ketoglutarate (HTK) solution or University of Wisconsin (UW) solution, Euro-Collins (EC), Hyperosmolar citrate (HOC, also known as Marshall's solution), Celsior solution, and Institut Georges Lopez-1 (IGL-1) solution. In some embodiments, the solution is low-potassium HTK. Custodiol HTK (histidine-tryptophan-ketoglutarate) cardioplegia solution (Custodiol; Koehler Chemi, Alsbach-Haenlien, Germany) (1 L) contains the following components: 15 mmol/L sodium chloride, 9 mmol/L potassium chloride, 4 mmol/L magnesium chloride, 18 mmol/L histidine hydrochloride, 180 mmol/L histidine, 2 mmol/L tryptophan, 30 mmol/L mannitol, 0.015 mmol/L calcium chloride, 1 mmol/L potassium hydrogen 2-ketoglutarate, osmolarity 310 mOsm/kg, pH 7.02-7.20. See, e.g., Bretschneider et al., J Cardiovasc Surg (Torino). 1975 May-June; 16(3):241-60.

Recovery Solution

A recovery solution for use in a method described herein is substantially the same as the sub-normothermic perfusion solution, without 3-OMG, including only 1-5%, e.g., 2-3% PEG, with an oxygen carrier. In some embodiments, the recovery solution is also hyperosmolar.

### Reduction of Liquid-Air Interfaces

Subzero non-frozen liquid (e.g., contained within a biological tissue sample) is intrinsically metastable and can spontaneously transform to lower-energy-level ice crystals through the formation of ice nuclei, which can be readily achieved by ice seeding. In the context of biological tissue sample preservation, formation of ice crystals is generally undesirable because of ice-mediated injury to cells (Bruinsma, B. G. & Uygun, K. Curr. Opin. Organ Transplant. 22, 281-286 (2017); Berendsen, T. A. et al. Nat. Med. 20, 790-793 (2014); Bruinsma, B. G. et al. Nat. Protoc. 10, 484-494 (2015)), which can cause cell death and organ damage.

In the context of subzero non-freezing preservation, liquid-air interfaces provide thermodynamically favorable sites of heterogeneous ice nucleation due to surface tension present at the interface. The present disclosure demonstrates that formation of ice crystals or ice nucleation can be reduced, e.g., significantly reduced, during high subzero preservation in the subzero non-freezing preservation phase, by reducing or eliminating liquid-air interfaces. For example, air can be removed from a storage solution bag that is holding a biological tissue sample (e.g., an organ) between stage 4 and stage 5, before subjecting the biological tissue sample to subzero non-freezing preservation in stage 5. Such air removal can be achieved by various methods, including immersing the storage solution bag containing the biological tissue sample in water or other liquid (i.e., water displacement method), which results in the water or other liquid pushing out the air in the bag, or using a vacuum pump to remove air from the storage solution bag. When using the vacuum methods, the container for the biological tissue sample can be rigid, whereas when using the displacement method, the container must be flexible. In some embodiments, and as mentioned elsewhere in the specification, all hair extending from a surface of an epidermis of a biological tissue sample (e.g., a VCA) can be removed, e.g., with depilatory chemical agents, for example, thereby reducing or eliminating liquid-air interfaces.

In some embodiments, the elimination of liquid-air interfaces can be performed after pre-conditioning the biological tissue sample with one or more perfusion solutions (e.g., after SNMP step), and prior to subzero non-freezing preservation.

### Machine Perfusion and Subzero Non-Freezing Preservation System

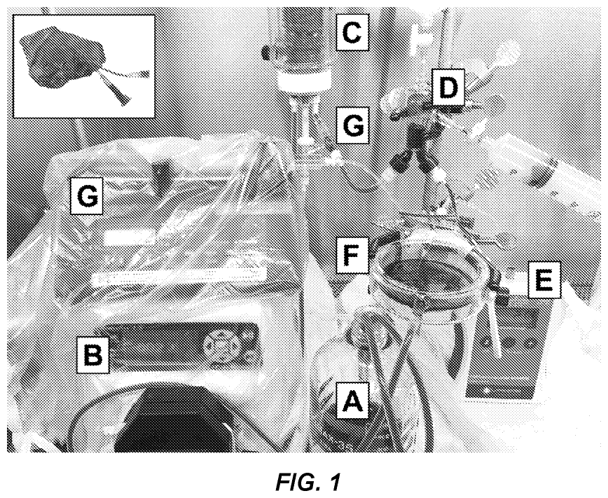

The present disclosure relates to machine perfusion systems that can perform the perfusion protocols described herein. The machine perfusion systems can include a pump (e.g., a roller pump) that is configured to produce flow, e.g., pulsatile or non-pulsatile flow (e.g., duplex non-pulsatile circulation), a perfusate reservoir (e.g., a jacketed organ chamber), a heat exchanger, a hollow fiber oxygenator, a jacketed bubble trap, a pressure sensor, and/or a sampling port. These components of the perfusion systems can be serially connected by a tubing (e.g., silicon tubing). In some embodiments, the perfusate and/or biological tissue sample temperature can be controlled by a separated warming/cooling circuits. The warming circuit can warmed by a warm water bath, while the cooling circuit can be cooled by a chiller. Both circuits can be pumped through heat exchanger and the jackets of the bubble t raps and the organ chamber. The chiller can include a refrigerant basin that can hold the biological tissue sample during subzero non-freezing preservation. An exemplary system is shown in FIG. 1, with a circuit consisting of perfusion solution (A) that is pumped via a roller pump (B) to the oxygenator (C), that is oxygenated with a carbogen mixture (5% CO2 and 95% oxygen). The solution then goes through the bubble trap (D) to prevent air bubbles going into the limb. The pressure is measured (E) at the level of the limb that is laying the basin (F). Inflow samples are measure at the inflow valve (G) with outflow samples are measured directly from the venous outflow canula (as shown in upper left panel).

In some embodiments, the machine perfusion and subzero non-freezing preservation system can be controlled by a computer control unit that is operatively connected to the other components of the system such that the computer control unit can control parameters such as perfusate temperature, perfusate flow rate, and time duration and sequence with which these parameters are maintained, to perform the perfusion protocols described herein.

### EXAMPLES

The invention is further described in the following examples, which do not limit the scope of the invention described in the claims.

### Methods

The following methods were used in the Examples below.

### 1. Perfusion Media

1.1. Muscle Media for Machine Perfusion of Vascularized Composite Allografts

An exemplary perfusion medium used for perfusion above 10 degrees Celsius is mainly based on a mixture of commercially available muscle media (PromoCell®) and an acellular oxygen carrier (HBOC-201, Hemopure®) (Table 1). While the perfusion of solid organs using a growth media is widely accepted (Williams Medium E for perfusion of livers and kidneys), to the best of the inventors' knowledge this is the first report of perfusion of vascularized composite allografts using a specific muscle media.

1.2. Growth Factors During Subnormothermic Machine Perfusion of Vascularized Composite Allografts

The PromoCell muscle media comes as a kit that consist of the media itself plus additives: Fetal Calf Serum, Fetuin, Insuline, Dexamethason and growth factors. These experiments only used the media itself with the addition of Epidermal Growth Factor (recombinant human) (10 ng/mL) and Basic Fibroblast Growth Factor (recombinant human) (1 mg/mL). The present methods include perfusion of vascularized composite allografts at non-physiological temperature (subnormothermic perfusion, 21 degrees Celsius) using growth factors to induce the growth of epidermal cells and fibroblasts.

1.3. Prostaglandin

Prostaglandin is a known vasodilator (38). We used the vasodilator prostaglandin during machine perfusion of vascularized composite allografts; the addition of prostaglandin via a microflow drip had a beneficial effect on the flow rate during machine perfusion. The combination of a perfusion solution based on HBOC-201 and muscle media in combination with prostaglandin greatly reduced the formation of edema during perfusion (Table 2). An increase in edema of more than 20% (over the course of the entire protocol) was associated with worse real-time perfusion parameters. The amount of edema was calculated as: Edema=weight^{end}−weight^{start}/weight^{start}*100%.

### 2. Perfusion Above 10 Degrees Celsius Technique

2.1. Surgical Technique

2.1.1. Systemic Heparin

During the donor procurement, heparinization of the graft is important to prevent blood clots within the graft. We found that systemic heparinization with 30 IU of heparin via the dorsal penile vein profoundly improved the flow in the beginning of perfusion (data not shown).

2.1.2. Anesthesia During the Donor Procedure

During surgery, isoflurane is used for the induction of anesthesia. We found it to very important to half the induction of dose prior to the microsurgery. If this was not done correctly, most animals died during surgery. The timing of turning down the isoflurane seems important, because during the preparation of the donor site (so prior to the microsurgery), the animals needed to full dose to stay adequately sedated.

2.2. Priming the Machine Perfusion System

Priming of the system was important for an accurate representation of the flowrate and to prevent perfusion mismatch due to are bubbles. This was especially important for the perfusion of rodent VCA, as the diameter of both the main vessels and the microvasculature is very small. A 24 gauge cannula was connected to the inflow outflet (a similar canula as used for the cannulation of the artery). The inflow cannula was secured to the based (sterile tape used if needed) at the level of the pressure valve in a 20-30 degree angle pointing downwards. The system was then be calibrated to the atmospheric pressure by pausing the roller pump, open the pressure valve and push the zero button on the pressure reader multiple times. This calibration process only works if the fluid is not in motion. The flowrate increased with increments of 0.1 mL/min circa every 2 minutes until a flowrate 2.0 mL/min is reached. During perfusion of the graft, the real time pressure was downed by the pressure that was noted during the priming process (so only the pressure of the fluid through the cannula without the VCA graft attached) to calculate the ‘real pressure’.

### 3. High Subzero Storage Between 0 to Minus 39 Degrees Celsius

3.1. Shaving and Hair Removal

During the ‘subzero non frozen state’ of the high subzero storage phase, the slightest impurity in the solution can trigger ice crystal formation. During the surgery, all VCA graft limbs were disinfected and shaved using an electrical trimmer used to animal shaving (1 mm). Still ice crystal formation occurred with these grafts. During surgery, we added an extra hair removal step by coating the skin with approximately 5 mL of NAIR (a chemical hair remover). The product was left on the skin for approximately 2-4 minutes and the hairs and residue were whipped off the graft using a sterile gauze. This extra step helped with minimizing micro vibrations on the surface of graft, thereby minimizing ice nucleation.

3.2. Micropump for Loading Subzero Non-Freezing Preservation Solution

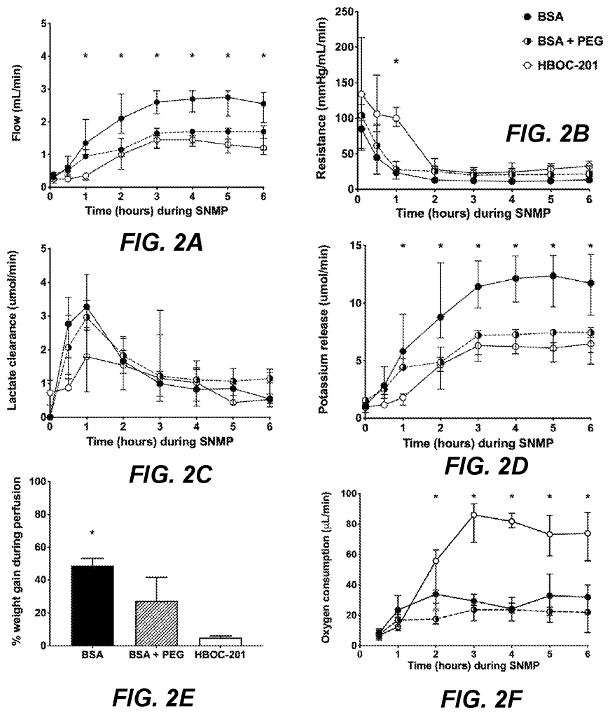

A micro syringe pump was used load the graft with the subzero non-freezing preservation solution prior to the high subzero preservation below 0 degrees Celsius. The micro syringe helped to gently perfuse the graft, without building up too much pressure thereby minimizing endothelial damage. We first perfused circa 6-7 mL of cold loading solution at a flow rate of 0.5 mL/min. ice. We then replaced the cold loading syringe by a syringe filled with subzero non-freezing preservation solution. The flowrate was then set to 0.2 mL/min and to perfuse the limb with the subzero non-freezing preservation solution for 30 minutes. After 30 minutes, the limb was disconnected from the syringe pump and placed in a sterilized mini organ bag with 15 mL of subzero non-freezing preservation solution evenly spread around the limb (FIG. 2).

3.3. Anti-Freeze Bath

Since vibrations of the chiller can also initiate ice crystallization, the grafts in this protocol were hung in the anti-freeze solution to buffer the vibrations (FIG. 8).

### Example 1: VCA Procurement

Twelve male Lewis rats (250-300 g) were used as hindlimb donors and muscle biopsies of another 3 male rats were used to set reference values (Charles Rivers Laboratories, Wilmington, Mass., USA). In part B, thirty-seven male Lewis rats (250-300 g) were used as hindlimb donors an another thirty-seven male Lewis rats (300-350 g) were used as transplant recipients. In all experiments, the right hindlimb was harvested as a model for an osteomyocutaneous VCA graft. Animals were housed and maintained in accordance with the National Research Council guidelines and the experimental protocol was approved by the Institutional Animal Care and Use Committee (IACUC) of the Massachusetts General Hospital (Boston, Mass., USA).

A heterotopic transplant model as previously described by Ulusal and colleagues was used (Ulusal A. E. et al. Heterotopic hindlimb allotransplantation in rats: an alternative model for immunological research in composite-tissue allotransplantation. Microsurgery 2005; 25:410-414), with the adjustment of not transplanting the foot to minimize automutilation and pressure ulcera. Animals were anesthetized using isoflurane (Forane, Baxter, Deerfield, Ill.) using a Tech 4 vaporizer (Surgivet, Waukesha, Wis.). Animals were placed on a heating pad in a supine position and were shaved from the right ankle with the distal lower ribs as the proximal and midline as the medial landmarks. The animals were prepped in a sterile manner using povidone iodine and surgical drape. The line on medial on medial side of the hindlimb overlies the femoral vessels and the circular lines on mid-thigh and above the ankle, respectively, delineate the skin paddle (4×3 cm). We started by a circular skin incision at the location of the medial above the ankle. First, the anterior and posterior tibial pedicles were ligated first using 8/0 ethilon sutures. The Achilles tendon was then sectioned, and the tibial periosteum was exposed by pushing back all tendons using a Obwegeser periosteal elevator. At this point, animals were systemically heparinized (30 IU) via the dorsal penile vein. For the skin paddle, we incised the line on the inner thigh. The fat pad was then dissected out to identify the femoral vessels and all surrounding muscle were cut off. Subsequently, both the femoral artery and vein were skeletonized and cannulated with a 24-gauge intravenous catheter that was secured with 7/0 silk ligation. The graft was mobilized by cutting the bones above the ankle and under the inguinal ligament and flushed with 10 mL heparinized saline (10 IU/mL) via the femoral artery till limpid outflow.

At this point, experimental limbs were wrapped in gauze and transferred to the perfusion system where perfusion was started within 10-15 minutes of warm ischemia time, while control limbs were flushed with 10 mL of University of Wisconsin (UW) solution and stored in a bag of 50 mL of UW solution on ice (4 degrees Celsius) referred to static cold storage (SCS). Moreover, to set a reference value for the energy charge analysis, which will be explained in more detail later on, muscle biopsies were collected from 3 anesthetized, untreated rats (in vivo controls).

### Example 2: Optimization of Perfusion Solution

### Experimental Groups

Three different perfusion solutions were tested for 6 hours of subnormothermic machine perfusion (SNMP) of rodent partial hindlimbs. A detailed overview of all perfusion solutions is summarized in Table 1. In all groups, skeletal muscle media with basic epidermal and fibroblast growth factors (PromoCell, C-23160, Heidelberg, Germany) provided the base of the solution. Bovine serum albumin (BSA) was the base colloid component in all groups. Also, additional supplements such as insulin, heparin, dexamethasone, hydrocortisone and antibiotics were similar between groups. The main differences between these perfusion solutions were based on the presence or absence of these 2 components: 1) Addition of polyethylene glycol (PEG) with a molecular weight of 35 kDa, and 2) Addition of an acellular oxygen carrier, HBOC-201 (Hemopure, HbO2, Therapeutics LLC) in combination with vasodilator prostaglandin.

Table 1 and Table 2 below provide an overview of the components of the various perfusion solutions. The total volume of the perfusion solution was 500 mL in all groups. Prior to connecting the limb, pH was optimized (pH 3.5-4.5) upon addition of bicarbonate.

TABLE 1
Overview of Sub-Normothermic Perfusion Solutions

| | Group 1 | Group 2 | Group 3 |
|---|---|---|---|
| | BSA | BSA + PEG | HBOC-201 |
| | n = 4 | n = 4 | n = 4 |
| Solution base | | | |
| PromoCell muscle media (mL) | 500 | 500 | 375 |
| HBOC-201 (mL) | — | — | 125 |
| Differentiating additives | | | |
| Bovine serum albumin (BSA) (g) | 10 | 10 | 10 |
| Polyethylene glycol (PEG) (g) | — | 15 | 15 |
| Prostaglandin¹ (μL/min) | — | — | 0.2 |
| Additional supplements | | | |
| Penicillin-Streptomycin (mL) | 2 | 2 | 2 |
| L-glutamine (mL) | 5 | 5 | 5 |
| Insulin (μL) | 100 | 100 | 100 |
| Heparin (mL) | 1 | 1 | 1 |
| Hydrocortisone (μL) | 100 | 100 | 100 |
| Dexamethasone (μg) | 8 | 8 | 8 |

Abbreviations used; BSA = bovine serum albumin, PEG = polyethylene glycol and HBOC-201 = hemoglobin-based oxygen carrier-201.
¹Prostaglandin is Alprostadil 500 mcg/mL vial is diluted in 50 mL of saline according to manufacturing instructions. This mixture was added to the solution via a syringe pump at a flow rate of 0.2 μL/min

TABLE 2
Testing Sub-Normothermic Machine Perfusion Solutions

| | | | | | | |
|---|---|---|---|---|---|---|
| Duration | SNMP | min | 360 | 360 | 360 | 360 |
| Solution | Williams | mL | 500 | 500 | // | // |
| | Medium E | | | | | |
| | Muscle medium | mL | // | // | 375 | 375 |
| | Hemopure | mL | // | // | 125 | 125 |
| Additives | PenStrep | mL | 2 | 2 | 2 | 2 |
| | L-Glutamine | mL | 5 | 5 | 5 | 5 |
| | Hydrocortisone | μL | 100 | 100 | 100 | 100 |
| | Insulin | μL | 100 | 100 | 100 | 100 |
| | Heparin | mL | 1 | 1 | 1 | 1 |
| | Dexamethasone | μg | 8 | 8 | 8 | 8 |
| | BSA | g | 5 | 7 | 10 | 10 |
| | PEG | g | 10 | 10 | 15 | 15 |
| | Bicarb | mg | 100 | 400 | 400 | 400 |
| Vasodilator | Prostaglandin | uL/min | 0.2 | 0.2 | 0.2 | 0.2 |
| | drip | | | | | |
| Weight gain | T = 0 | mg | 20 | 18.9 | 21.8 | 22.7 |
| | T = end | mg | 25.3 | 28.5 | 23.2 | 23.8 |
| | % | | 26.5 | 50.8 | 6.4 | 4.8 |

Abbreviations used; SNMP = subnormothermic machine perfusion. BSA = bovine serum albumin, PEG = polyethylene glycol and HBOC-201 = hemoglobin-based oxygen carrier-201.

### Hemodynamic Parameters

Arterial flow increased in all groups during the first half of perfusion and remained stable thereafter (FIG. 2A). After 1 hour of SNMP, median flows were significantly higher in the BSA group compared to the HBOC-201 group, 1.4 (1-2.1) vs. 0.4 (0.2-0.4) mL/min (p=0.01) respectively. Median flows continued to be higher in the BSA group compared to the HBOC-201, but not the BSA+PEG, group until the end of 6 hours perfusion, 2.6 (2.0-2.9) vs. 1.2 (1.0-1.5) mL/min respectively (p=0.04).

Vascular resistance decreased in all groups during the first hour of perfusion and remained stable thereafter (FIG. 2B). After 1 hour of SNMP, median vascular resistance was significantly higher in the HBOC-201 compared to the BSA, but not BSA+PEG, group, 100.4 (88.8-115.2) vs. 23.5 (14.6-24.8) mmHg/mL/min (p=0.02).

### Example 3: Machine Perfusion System

For 6 hours of SNMP, we used a self-built machine perfusion system (see FIG. 1). Key components for the system were a rotating pump (07522-20 DRIVE MFLEX L/S 600 RPM 115/230, Cole-Parmer, Vernon Hills, Ill.), tubing (Mastedlex platinum-cured silicone tubing, L/S 16, Cole-Parmer, Vernon Hills, Ill.) and a membrane oxygenator, bubble trap chamber and tissue bath (catalog numbers 130144, 130149 and 158400 respectively, Radnoti LTD, Dublin, Ireland). Vascular pressure was measured via a pressure transducer (PT-F, Living Systems Instrumentation, St Albans City, Vt.) and read by a portable pressure monitor (PM-P-1, Catamount Research and Development, St Albans, Vt.). Prior to connecting the limb, pressures of the system without the limb were noted at different flow rates (Pressurewithout). During perfusion, pressures with the limb were observed (Pressurewith) and flows were adjusted accordingly to aim for a vascular pressure between 30-40 mmHg. The vascular pressure was calculated as Pressurewith−Pressurewithout. Vascular resistance was calculated by dividing the vascular pressure by the flow rate.

### Example 4: Biological Tissue Sample Viability Analysis

### Perfusion Samples and Muscle Biopsies

During 6 hours of perfusion, perfusion samples were collected from both the arterial inflow and venous outflow. An i-STAT analyzer (Abbott, Princeton, N.J.) was used to measure perfusate levels of potassium and lactate as well as oxygen tension and saturation. At the end of 6 hours of SNMP, biopsies form the m. rectus femoris were taken. Biopsies were snap-frozen in liquid nitrogen and stored in a −80 degrees Celsius freezer for mass spectrometry or stored in formalin for histological analysis.

### Perfusate Injury Markers

Lactate clearance (μmol/min) was calculated by the difference between the arterial and venous lactate concentration (mmol/L) and corrected for flow (mL/min). Potassium release (μmol/min) was calculated as differences in concentration (mmol/L) between the arterial inflow and venous outflow and corrected for flow (mL/min).

### Oxygen Consumption

Total oxygen consumption was calculated by the difference between the arterial and venous oxygen content and corrected for flow. The following formula was used for calculations: total oxygen consumption (μL 02/min)=cO₂*(pO₂^{art-ven}*flow)+(Hb+cHb+(SO₂^{art-ven}*flow)), where cO2 is the oxygen solubility coefficient (3.14*10⁻⁵ mLO2/mmHg O2/mL), pO₂^{art-ven} is the difference in partial oxygen pressure between in artery inflow and venous outflow (mmHg), flow is the arterial inflow (mL/min), Hb is the hemoglobin concentration (g/mL) and cHb is the oxygen binding capacity of Hb (1.26 for HBOC-201).

In all groups, lactate clearance increased within the first hour of perfusion and declined thereafter, as presented in FIG. 2C. During 6 hours of perfusion, there was no statistical difference in lactate clearance between the groups. However, it must be noted that the HBOC-201 perfusion fluid had a median lactate concentration of 2.9 mmol/L (2.9-3.0) prior to perfusion while the BSA and BSA+PEG had unmeasurable concentration of lactate prior to perfusion. This can be explained by the presence of sodium lactate (27 mmol/L) in the HBOC-201 solution as described in the product sheet by the manufacturer (Hemopure, HbO2, Therapeutics LLC). During the 3 hours of SNMP, potassium concentration increased in all groups but levels stabilized thereafter, as presented in FIG. 2D. After 1 hour of SNMP, median potassium release was significantly higher in the BSA group compared to the BSA+PEG and HBOC-201 group, 5.8 (4.3-9.0) vs. 4.4 (4.2-5.2) vs. 1.8 (1.1-2.1) μmol/min (p=0.005) respectively. Potassium release continued to be significantly higher in the BSA group compared to the BSA+PEG and HBOC-201 group for the remainder of 6 hours of SNMP. After 6 hours of SNMP, median potassium release was in the was significantly higher in the BSA group compared to the BSA+PEG and HBOC-201 group, 11.7 (9.0-14.2) vs. 7.4 (5.7-7.9) vs. 6.5 (4.7-7.1) μmol/min respectively (p=0.003).

After 2 hours of SNMP, total oxygen consumption was significantly higher in the HBOC-201 group compared to the BSA and BSA+PEG group, 55.8 (27.7-63.0) vs. 33.9 (24.0-36.6) vs. 17.5 (14.3-23.0) μL/min respectively (p=0.033) (FIG. 2F). While oxygen consumption stabilized after the first 2 hours in the BSA and BSA+PEG group, oxygen consumption in the HBOC-201 continued to increase during the first 3 hours of SNMP before it stabilized for the remainder of SNMP (FIG. 2F). After 3 hours of SNMP, total oxygen consumption was significantly higher in the HBOC-201 group compared to the BSA and BSA+PEG group, 86.1 (68.1-93.3) vs. 29.4 (26.5-33.8) vs. 23.7 (16.3-26.6) respectively μL/min (p=0.005). At the end of 6 hours SNMP, total oxygen consumption continued to be significantly higher in the HBOC-201 group compared to the BSA and BSA+PEG group, 74.0 (55.8-87.8) vs. 31.9 (21.5-40.0) vs. 22.0 (8.6-31.5) μL/min respectively (p=0.023).

### Weight Gain Due to Edema

Limbs were weighed prior to and after 6 hours of SNMP. Median start weight of all limbs (prior to perfusion) was 19 (17-21) grams and did not differ between groups (p=0.11). Median weight gain (as a percentage of baseline) was significantly lower in the HBOC-201 group with an increase of 4.9 (4.3-6.1) percent compared to limbs perfused with BSA alone or BSA+PEG, 48.8 (39.1-53.2) and 27.3 (20.5-41.6) percent respectively (p=0.005) (FIG. 2E).

### Energy Charge Analysis

After 6 hours of either SNMP or SCS, muscle biopsies were taken and snap frozen in liquid nitrogen. All muscle biopsies were analyzed with liquid chromatography-mass spectrometry for energetic cofactors (adenosine triphosphate [ATP]/adenosine diphosphate [ADP]/adenosine monophosphate [AMP]), referred to as energy charge. Preserved energy status appears critical for post-transplant outcome (18).

All frozen tissue biopsies were pulverized, weighted (averaging circa 25 mg) and analyzed for energetic cofactors using targeted multiple reaction monitoring (MRM) analysis on a 3200 triple quadrupole liquid chromatography-mass spectrometry (QTRAP LC/MS-MS) system (AB Sciex, Foster City, Calif.), as previously described (18). In short, metabolites were extracted using a mixture of methanol/chloroform, followed by 3 freeze-thaw cycles. Each extract was then diluted with ice-cold water (200 μL), centrifuged for 1 minute at 15 000×g before the top layer was transferred to an autosampler vial for mass spectrometry analysis. In this study, MRM transitions for ATP, ADP and AMP were quantified and energy charge was calculated as; Energy Charge=(ATP+0.5*ADP)/(ATP+ADP+AMP).

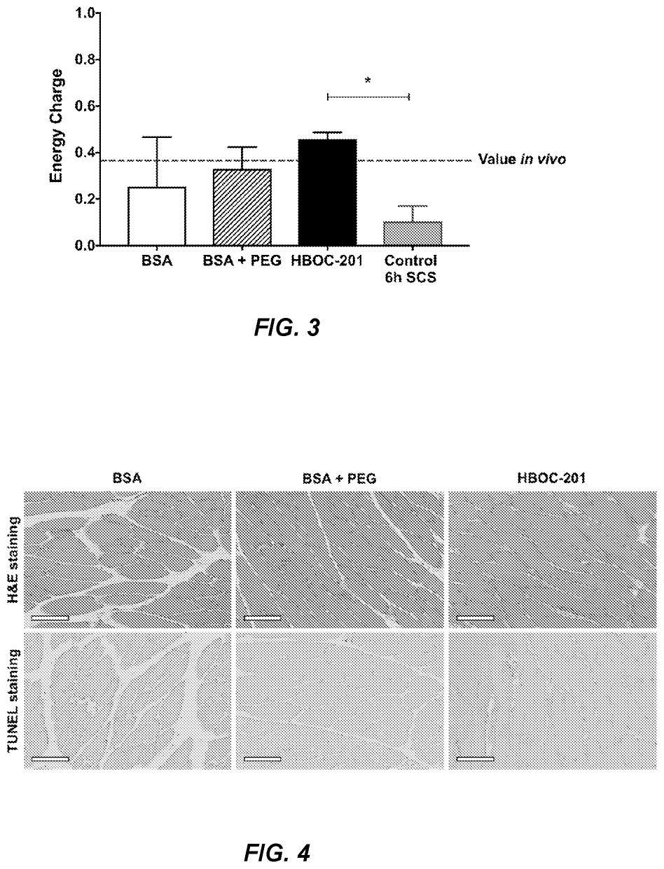

Energy charge ratios are summarized in FIG. 3. At the end of 6 hours of perfusion, median energy charge rations were comparable between the BSA, BSA+PEG and HBOC-201 groups, 0.25 (0.15-0.47) vs. 0.33 (0.23-0.42) vs. 0.46 (0.42-0.49) (p=0.20) respectively. Interestingly, all energy charge ratios of all groups were comparable to the energy charge ratio of in vivo controls (median ratio 0.37 (0.19-0.58)), as indicated by the red dotted line in FIG. 3. However, energy charge ratios of SCS control limbs were significantly lower compared to HBOC-201 perfused limbs, but not BSA and BSA+PEG limbs, 0.10 (0.07-0.17) vs. 0.46 (0.42-0.49) (p=0.002) respectively.

### Histology Analysis of Muscle Biopsies

Muscle biopsies were fixated in formalin, paraffin embedded, and cross-sectioned. Slides were stained with hematoxylin and eosin (H&E) and apoptosis marker TUNEL by the pathology department of our center. After staining, all biopsies were digitally captures using bright microscope and structural myocyte injury was assessed.

None of the muscle biopsies showed myocyte injury or degeneration after perfusion. Furthermore, none of the muscle biopsies showed apoptotic cell death. Biopsies of BSA perfused limbs showed, however, more signs of interstitial edema compared to HBOC-201 perfused limbs (FIG. 4).

### Example 5: Optimal Perfusion Group

As previously described in part A, the HBOC-2019 perfusion group proved to be superior compared to the other perfusion groups in terms of edema, oxygen delivery and energy charge. We therefore chose to validate the HBOC-2019 group in a transplant setting. In total, twenty right partial hindlimbs were transplanted were transplanted after 6 hours of SNMP (HBOC-201 group). Transplant controls included hindlimbs that were preserved for 6 hours of SCS (n=4), 24 hours of SCS (n=5) or hindlimbs that were transplanted directly after harvest referred to as fresh controls (n=8).

### Example 6: Transplantation

Heterotopic hindlimb transplantation was performed as described previously by Ulusal and colleagues (17). Transplant recipients were all male Lewis rats (300-350 g). Anesthesia of the recipient was performed in a similar fashion as during the donor procedure and the rat was positioned on the lateral right to expose the left hip area. Recipients received 0.05 mg/kg buprenorphine hydrochloride subcutaneously prior to incision. An inguinal incision was made to expose the fat pad and the femoral vessels. A subcutaneous pocket was created to inset the donor graft, on the dorsal side of the rat, the skin was undermined from the inguinal incision towards the groin area. Depending of the vessel length, mobility and graft size, a dorsal skin incision was made. The skin of the donor graft was fixed circumferentially to the adjacent skin with absorbable vicryl 6/0 stiches. The donor pedicle was placed in in the subcutaneous tunnel towards the recipient vessels. Recipient vessels were ligated proximal to the epigastric vessels and an end-to-end microvascular anastomosis of the donor and recipient artery and vein was performed using 10/0 sutures. The inguinal incision was closed using absorbable vicryl 6/0.

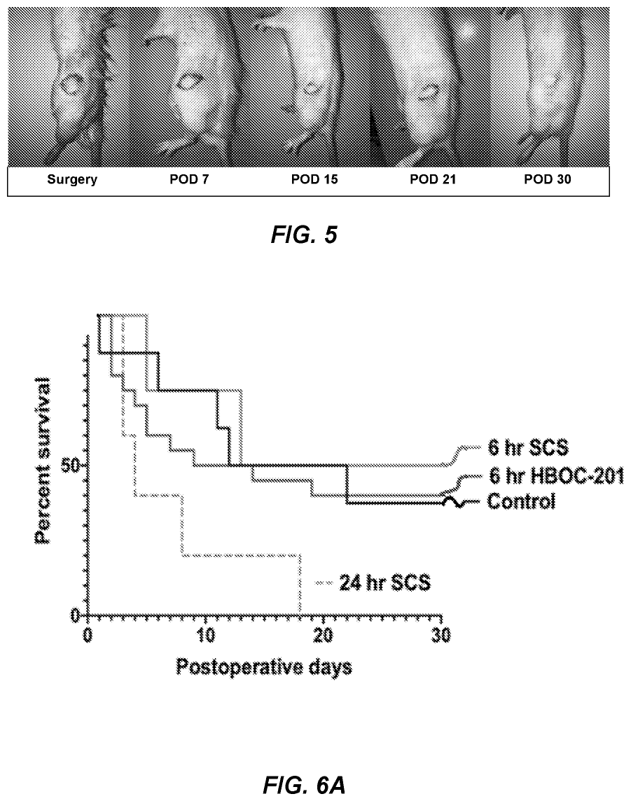

All transplant recipients were followed for 30 days (FIG. 5). Automutilation by the animals was a major concern in this study. In both the fresh control group and the HBOC-201 group 20% of the animals had to be terminated early because of signs of automutilation. These animals were not included in the survival analysis.

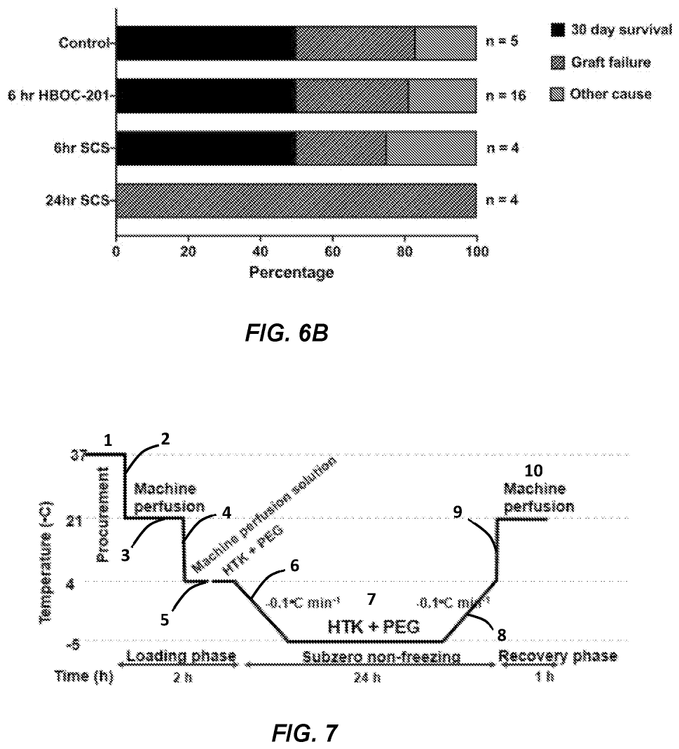

Overall survival in the group that received a graft that was perfused for 6 hours with the HBOC-201 protocol was 50%, which was similar to the control group that received an untreated, fresh graft (also 50% survival) (FIG. 6A). Moreover, animals that received a graft that was preserved with 6 hours of SCS also had a survival of 50% after 30 days. Negative controls that received a graft that was preserved using 24 hours of SCS did not survive past day 8.

Mortality rates due to graft failure were 33% in the control group, 31% in the HBOC-201 group and 25% in the SCS group whilst in the negative control group (24 hours SCS) all animals died because of graft failure (100%) (p=0.89) (FIG. 6B). Other reasons of death included death because of too much intra-operative blood loss, hypothermia or stress.

### Post-Operative Management

Post-operative follow up was 30 days in all study groups. During the first 72 hours after transplantation, graft recipients received 0.05 mg/kg Buprenex® subcutaneously every 12 hours. The recipients and their grafts were inspected twice a day. Viability of the graft was assessed by physical examination: temperature (cold or body temperature), color (pale or blue) and turgor (swelling). In case of suspected graft failure based on the physical examination, the recipient was terminated immediately. Also, if during the post-operative follow up rats showed signs of automutilation, wounds or infection, the rats were terminated before the end of the study in accordance with the National Research Council guidelines and Institutional Animal Care and Use Committee (IACUC) of the Massachusetts General Hospital (Boston, Mass., USA).

### Statistical Analysis

Continuous data are reported as medians with interquartile range, categorial variables as absolute numbers. Differences between groups were analyzed using a Kruskal-Wallis H test with a Dunn's post-test or Mann-Whitney test when applicable. All statistical analysis was performed using Prism 5.0a for Mac OSX (GraphPad Software, La Jolla, Calif.). P values less than 0.05 were considered to be significant.

### REFERENCES

1. Burlage L C, Tessier S N, Etra J W, Uygun K, Brandacher G. Advances in machine perfusion, organ preservation, and cryobiology: potential impact on vascularized composite allotransplantation. Curr Opin Organ Transplant 2018; 23:561-567.
2. Kueckelhaus M, Fischer S, Seyda M, et al. Vascularized composite allotransplantation: current standards and novel approaches to prevent acute rejection and chronic allograft deterioration. Transpl Int 2016; 29:655-662.
3. Blaisdell F W. The pathophysiology of skeletal muscle ischemia and the reperfusion syndrome: a review. Cardiovasc Surg 2002; 10:620-630.
4. Landin L, Cavadas P C, Garcia-Cosmes P, Thione A, Vera-Sempere F. Perioperative ischemic injury and fibrotic degeneration of muscle in a forearm allograft: functional follow-up at 32 months post transplantation. Ann Plast Surg 2011; 66:202-209.
5. Murata S, Miniati D N, Kown M H, et al. Superoxide dismutase mimetic m40401 reduces ischemia-reperfusion injury and graft coronary artery disease in rodent cardiac allografts. Transplantation 2004; 78:1166-1171.
6. Panizo A, Pardo F J, Lozano M D, de Alava E, Sola I, Idoate M A. Ischemic injury in posttransplant endomyocardial biopsies: immunohistochemical study of fibronectin. Transplant Proc 1999; 31:2550-2551.
7. Ozer K, Rojas-Pena A, Mendias C L, Bryner B S, Toomasian C, Bartlett R H. The Effect of Ex Situ Perfusion in a Swine Limb Vascularized Composite Tissue Allograft on Survival up to 24 Hours. J Hand Surg Am 2016; 41:3-12.
8. Werner N L, Alghanem F, Rakestraw S L, et al. Ex Situ Perfusion of Human Limb Allografts for 24 Hours. Transplantation 2017; 101:e68-e74.
9. Kueckelhaus M, Fischer S, Sisk G, et al. A Mobile Extracorporeal Extremity Salvage System for Replantation and Transplantation. Ann Plast Surg 2016; 76:355-360.
10. Berendsen T A, Bruinsma B G, Lee J, et al. A simplified subnormothermic machine perfusion system restores ischemically damaged liver grafts in a rat model of orthotopic liver transplantation. Transplant Res 2012; 1:6.
11. Bruinsma B G, Yeh H, Ozer S, et al. Subnormothermic machine perfusion for ex vivo preservation and recovery of the human liver for transplantation. Am J Transplant 2014; 14:1400-1409.
12. Bruinsma B G, Avruch J H, Weeder P D, et al. Functional Human Liver Preservation and Recovery by Means of Subnormothermic Machine Perfusion. J Vis Exp 2015.
13. Xu X, Philip J L, Razzaque M A, Lloyd J W, Muller C M, Akhter S A. High-molecular-weight polyethylene glycol inhibits myocardial ischemia-reperfusion injury in vivo. J Thorac Cardiovasc Surg 2015; 149:588-593.
14. Bejaoui M, Pantazi E, Folch-Puy E, et al. Protective Effect of Intravenous High Molecular Weight Polyethylene Glycol on Fatty Liver Preservation. Biomed Res Int 2015; 2015:794287.
15. Bejaoui M, Pantazi E, Calvo M, et al. Polyethylene Glycol Preconditioning: An Effective Strategy to Prevent Liver Ischemia Reperfusion Injury. Oxid Med Cell Longev 2016; 2016.
16. Matton A P M, Burlage L C, van Rijn R, et al. Normothermic machine perfusion of donor livers without the need for human blood products. Liver Transpl 2018; 24:528-538.
17. Ulusal A E, Ulusal B G, Hung L-M, Wei F C. Heterotopic hindlimb allotransplantation in rats: an alternative model for immunological research in composite-tissue allotransplantation. Microsurgery 2005; 25:410-414.
18. Bruinsma B G, Avruch J H, Sridharan G V, et al. Peritransplant energy changes and their correlation to outcome after human liver transplantation. Transplantation 2017; 101:1637-1644.
19. Vajdová K, Graf R, Clavien P-A. ATP-supplies in the cold-preserved liver: A long-neglected factor of organ viability. Hepatology 2002; 36:1543-1552.
20. Berendsen T A, Izamis M L, Xu H, et al. Hepatocyte viability and adenosine triphosphate content decrease linearly over time during conventional cold storage of rat liver grafts. Transplantation proceedings 2011; 43:1484-1488.
21. Hautz T, Hickethier T, Blumer M J F, et al. Histomorphometric evaluation of ischemia-reperfusion injury and the effect of preservation solutions histidine-tryptophan-ketoglutarate and University of Wisconsin in limb transplantation. Transplantation 2014; 98:713-720.
22. Azari K, Imbriglia J, Goitz R, et al. Technical Aspects of the Recipient Operation in Hand Transplantation. Journal of Reconstructive Microsurgery 2012; 28:27-34.
23. Kaufman C L, Marvin M R, Chilton P M, et al. Immunobiology in VCA. Transplant International 2016; 29:644-654.
24. Mundinger G S, Narushima M, Hui-Chou H G, et al. Infrared fluorescence imaging of lymphatic regeneration in nonhuman primate facial vascularized composite allografts. Ann Plast Surg 2012; 68:314-319.
25. Belzer F O, Southard J H. Principles of solid-organ preservation by cold storage. Transplantation 1988; 45:673-676.
26. Troutman E C. Hypothermic Machine Perfusion of Composite Tissues. :62.
27. Fichter A M, Ritschl L M, Borgmann A, et al. Development of an Extracorporeal Perfusion Device for Small Animal Free Flaps. PLoS One 2016; 11: e0147755.
28. Muller S, Constantinescu M A, Kiermeir D M, et al. Ischemia/reperfusion injury of porcine limbs after extracorporeal perfusion. J Surg Res 2013; 181:170-182.
29. Constantinescu M A, Knall E, Xu X, et al. Preservation of amputated extremities by extracorporeal blood perfusion; a feasibility study in a porcine model. J Surg Res 2011; 171:291-299.
30. Consoli A, Nurjhan N, Reilly J J J, Bier D M, Gerich J E. Contribution of liver and skeletal muscle to alanine and lactate metabolism in humans. Am J Physiol 1990; 259:E677-684.
31. Ahlborg G, Wahren J, Felig P. Splanchnic and peripheral glucose and lactate metabolism during and after prolonged arm exercise. J Clin Invest 1986; 77:690-699.
32. Bangsbo J, Aagaard T, Olsen M, Kiens B, Turcotte L P, Richter E A. Lactate and H+ uptake in inactive muscles during intense exercise in man. J Physiol 1995; 488 (Pt 1):219-229.
33. Fontes P, Lopez R, van der Plaats A, et al. Liver preservation with machine perfusion and a newly developed cell-free oxygen carrier solution under subnormothermic conditions. Am J Transplant 2015; 15:381-394.
34. Laing R W, Bhogal R H, Wallace L, et al. The Use of an Acellular Oxygen Carrier in a Human Liver Model of Normothermic Machine Perfusion. Transplantation 2017; 101:2746-2756.
35. de Vries Y, van Leeuwen O B, Matton A P M, Fujiyoshi M, de Meijer V E, Porte R J. Ex situ normothermic machine perfusion of donor livers using a haemoglobin-based oxygen carrier: a viable alternative to red blood cells. Transplant International 2018; 31:1283-1284.
36. Kan P, Miyoshi H, Ohshima N. Perfusion of medium with supplemented growth factors changes metabolic activities and cell morphology of hepatocyte-nonparenchymal cell coculture. Tissue Eng. 2004 October; 10(9-10):1297-307.
37. Rigo F, De Stefano N, Navarro-Tableros V, David E, Rizza G, Catalano G, et al. Extracellular Vesicles from Human Liver Stem Cells Reduce Injury in an Ex Vivo Normothermic Hypoxic Rat Liver Perfusion Model. Transplantation. 2018 May; 102(5):e205-10.
38. Guarrera J V, Polyak M M R, Arrington B, Boykin J, Brown T, Jean-Jacques M A, et al. Pushing the envelope in renal preservation; improved results with novel perfusate modifications for pulsatile machine perfusion of cadaver kidneys. Transplant Proc. 2004 June; 36(5): 1257-60.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for preserving a biological tissue sample, the method comprising:
(a) perfusing the biological tissue sample with a hyperosmolar sub-normothermic perfusion solution comprising one or more cryoprotective agents, one or more oxygen carrier agents, one or more growth factors, and one or more vasodilators, at a sub-normothermic temperature;
(b) perfusing the biological tissue sample with a subzero non-freezing preservation solution comprising at least one or more cryoprotective agents, at a hypothermic temperature;
(c) optionally placing the perfused biological tissue sample in a container and sealing the container; and
(d) cooling the biological tissue sample in the container to a subzero temperature without freezing the sample, thereby preserving the biological tissue sample at the subzero temperature.

2. The method of claim 1, further comprising:
(e) warming the biological tissue sample to a hypothermic temperature;
(f) perfusing the biological tissue sample with a recovery solution comprising one or more cryoprotective agents and one or more oxygen carrier agents at a sub-normothermic temperature; and
(g) warming the biological tissue sample to a normothermic temperature, thereby recovering the preserved biological tissue sample for use.

3. The method of claim 1, wherein the method further comprises,
preferably prior to step (a):
removing hair from the biological tissue sample, sufficient to avoid ice crystal formation within the biological tissue sample or the perfusion solution, optionally wherein the hair is removed from the biological tissue sample by contacting the biological tissue sample with a chemical depilatory agent.

4. (canceled)

5. The method of claim 1, wherein the sub-normothermic perfusion solution comprises one or more cryoprotective agents selected from polyethylene glycol (PEG) and 3-OMG, in a skeletal muscle cell growth medium.

6. The method of claim 1, wherein the hypothermic temperature is between 0° C. and 12° C.

7. The method of claim 6, wherein the hypothermic temperature is about 4° C.

8. The method of claim 1, wherein the sub-normothermic temperature is between 12° C. and 35° C.

9. The method of claim 9, wherein the sub-normothermic temperature is about 21° C.

10. The method of claim 1 wherein the normothermic temperature is between about 35° C. and 40° C.

11. The method of claim 1, wherein the normothermic temperature is about 37° C.

12. The method of claim 1, wherein the subzero temperature is about −4° C. or is below about −4° C.

13. (canceled)

14. The method of claim 1, wherein the methods comprise removal of sufficient air from the container to result in elimination or reduction of one or more liquid-air interfaces in the container, thereby reducing or eliminating formation of ice crystals.

15. The method of claim 1, wherein the biological tissue sample remains unfrozen when cooled to a subzero temperature.

16. The method of claim 1, wherein the biological tissue sample is a vascular composite allograft.

17. The method of claim 16, wherein the vascular composite allograft is a donor vascular composite allograft for vascular composite allograft transplantation.

18. The method of claim 1, wherein the biological tissue sample is obtained from a human, a primate, or a pig.

19. The method of claim 16, wherein the vascular composite allograft is at least a portion of a limb, face, larynx, trachea, abdominal wall, genitourinary tissue, uterine tissue, or solid organ, or a combination thereof.

20. The method of claim 2, wherein the recovery solution comprises one or more of polyethylene glycol (PEG), an oxygen carrier agent, a prostaglandin, an albumin, skeletal muscle cell growth medium.

21. The method of claim 1, wherein the sub-normothermic perfusion solution and the recovery solution comprise:
between 50 mL and 200 mL oxygen carrier agent per 500 mL;
between 1 g and 20 g of an oncotic agent, preferably albumin, per 500 mL;
between 1 g and 50 g 35 kDa of PEG per 500 mL;
between 0.02 μL/min and 2 μL/min prostaglandin (10 μg/mL); and
skeletal muscle cell growth medium.

22. (canceled)

23. The method of claim 1, wherein the sub-normothermic perfusion solution and the recovery solution comprise:
between 50 U and 150 μL insulin per 500 mL;
between 1 mg and 20 mg dexamethasone per 500 mL;
between 0.1 mL and 5 mL heparin per 500 mL;
between 1 mL and 10 mL antibiotic, optionally penicillin-streptomycin (5000 U/ml) per 500 mL;
between 1 mL and 10 mL L-glutamine per 500 mL; and
between 50 μL and 150 μL immune suppressant, optionally hydrocortisone 500 mL.

24. (canceled)

25. The method of claim 1, wherein:
steps (a) and (b), combined, are performed for a duration of approximately 2 hours; steps (d) and (e), combined, are performed for a duration of approximately 24 hours; and/or step (f) is performed for a duration of approximately 1 hour.

26. (canceled)

27. (canceled)

28. The method of claim 1, wherein the biological tissue sample is preserved at the subzero temperature for more than 12 hours.

29. The method of claim 2, wherein the biological tissue sample is viable after being recovered from subzero preservation, as determined by measuring one or more of a tissue adenosine triphosphate (ATP) to adenosine monophosphate (AMP) ratio, a tissue ATP to adenosine diphosphate (ADP) ratio, lactate levels, potassium concentration, terminal deoxynucleotidyl transferase dUTP nick end labeling (TUNEL), swelling and weight gain, percentage of edema, vascular resistance, oxygen consumption, lactic acid dehydrogenase (LDH) levels, and ischemia.

30. The method of claim 1, wherein the sub-normothermic perfusion solution and the recovery solution comprise a growth factor.

31. The method of claim 5, wherein the oxygen carrier agent is an acellular oxygen carrier agent or a cellular oxygen carrier.

32. The method of claim 31, wherein the acellular oxygen carrier agent is a hemoglobin-based oxygen carrier (HBOC) or a perfluorocarbon-based oxygen carrier (PFC).

33. (canceled)

34. A system for subzero preserving a biological tissue sample, the system comprising:
a pump;
a solution reservoir;
a heat exchanger;
a hollow fiber oxygenator;
a jackted bubble trap;
a pressure sensor;
a tubing that serially connects the pump, the solution reservoir, the heat exchanger, the hollow fiber oxygenator, the jacketed bubble trap, and the pressure sensor;
and a computer control unit that operates the system to perform any of the perfusion steps described in claim 1.

35.-43. (canceled)
